(19) United States
(12) Patent Application Publication
(10) Pub. No.: US 2024/0271073 A1
(43) Pub. Date: Aug. 15, 2024

(54) CELL CULTURE VESSEL FOR USE IN MANUFACTURING CELL PRODUCTS

(71) Applicant: Geoffrey L. Hodge, Sutton, MA (US)

(72) Inventor: Geoffrey L. Hodge, Sutton, MA (US)

(21) Appl. No.: 18/566,827

(22) PCT Filed: Jun. 7, 2022
(86) PCT No.: PCT/US22/32426

§ 371 Date: Dec. 4, 2023

Related U.S. Application Data
(60) Provisional application No. 63/197,665, filed on Jun. 7, 2021.

(30) Foreign Application Priority Data
Aug. 26, 2021 (EP) 21193332.0

(51) Int. Cl. C12M 1/00; C12M 1/04; C12M 1/34

(52) U.S. Cl. CPC C12M 23/34; C12M 23/24; C12M 41/14; C12M 41/44

(57) ABSTRACT

An automated manufacturing system and method of manufacture of a cell therapy are disclosed herein. In some embodiments, the system includes one or more workstations for processing a cell culture, with the cell culture being moved between workstations in a cell culture vessel. In some embodiments, the cell culture vessel includes an inner container (350) and an outer shell (358). In some embodiments, the shell includes a top (374) and bottom (376) that cooperate with one another to form a chamber for holding the inner container.

### CROSS-REFERENCE TO RELATED APPLICATIONS

This application claims the benefit of, and priority to, U.S. Provisional Application No. 63/197,665 titled “Cell Culture Vessel For Use In Manufacturing Cell Products”, filed on Jun. 7, 2021, and European Patent Application No. EP21193332.0, titled “Cell Culture Vessel For Use In Manufacturing Cell Products”, filed on Aug. 26, 2021, the disclosures of each of which are herein incorporated by reference in its entirety.

### FIELD

This application relates generally to cell culture vessels, and more particularly to cell culture vessels for use in a manufacturing system for preparing a cell therapy.

### BACKGROUND

Cancer immunotherapy, including cell-based therapies, antibody therapies, and cytokine therapies, are used to provide immune responses attacking tumor cells while sparing normal tissues. Cell-based therapies may be prepared by obtaining immune cells for a subject to be treated, genetically engineering the cells, and administering the cells to the subject for treatment. Such cellular therapies, especially if individualized for each patient, are complex and expensive in manufacturing, as cells need to be grown, certain parameters of the tissue culture determined and manipulated in various steps. Optimized cell culture vessels are therefore required for easier handling and especially for automated manufacturing.

### SUMMARY

The present disclosure is based, at least in part, on the development of cell culture vessels suitable for use in automated manufacturing processes to produce cell therapies. Also provided herein are methods for manufacturing cell therapies using the cell culture vessels disclosed herein. Such methods can be performed in automated manners.

In one embodiment, a cell culture vessel is disclosed, which includes an inner container having a pocket defining a volume within which a cell culture is maintained during manufacture of a cell therapy, and an outer shell arranged to receive and support the container, wherein the outer shell includes a shell top and a shell bottom that cooperate with one another to form a chamber within which the inner container is disposed, optionally, encapsulated.

According to another embodiment a method of manufacturing one or more cell therapies using a system having an incubator and one or more workstations is disclosed. The method includes moving a cell culture vessel to a first workstation, wherein the cell culture vessel comprises an inner container having a pocket defining a volume within which a cell culture is maintained during manufacture of a cell therapy and an outer shell arranged to receive and support the inner container, wherein the outer shell includes a shell top and a shell bottom that cooperate with one another to form a chamber within which the inner container is disposed, optionally, encapsulated, at least one of separating the cell culture, analyzing the cell culture, and transferring liquid into and out of the cell culture vessel, and removing the cell culture vessel from the first workstation. It should be appreciated that the foregoing concepts, and additional concepts discussed below, may be arranged in any suitable combination, as the present disclosure is not limited in this respect.

In another embodiment, a system for manufacturing cell therapies is disclosed. The system includes an incubator arranged to house first, second, and third cell culture vessels with first, second, and third cell cultures, respectively, one or more analysis workstations, one or more separation workstations, and one or more liquid addition workstations. Each of the first, second, and third cell cultures is arranged to be processed by at least one of the one or more analysis workstations, the one or more separation workstations, and the one or more liquid addition workstations before being returned to the incubator.

According to another embodiment, a method of manufacturing one or more cell therapies is disclosed. The system includes one or more analysis workstations, one or more separation workstations, and one or more liquid addition workstations. The method includes removing a first cell culture vessel holding a first cell culture from an incubator, the incubator arranged to house first, second, and third cell culture vessels, moving the first cell culture vessel to the one or more analysis workstations, analyzing the first cell culture at the one or more analysis workstations, moving the first cell culture vessel to at least one of the one or more separation workstations and the one or more liquid addition workstations, processing the first cell culture at the at least one of the one or more separation workstations and the one or more liquid addition workstations, and returning the first cell culture vessel to the incubator.

The foregoing and other aspects, embodiments, and features of the present teachings can be more fully understood from the following description in conjunction with the accompanying drawings.

### BRIEF DESCRIPTION OF THE DRAWINGS

Various embodiments of the invention will now be described, by way of example, with reference to the accompanying drawings, in which:

FIG. 1 is a schematic representation of a manufacturing system for a cell therapy according to embodiments disclosed herein;

FIG. 2 illustrates a method of preparing a cell therapy according to some embodiments;

FIG. 3 is an inner container of a cell culture vessel according to embodiments disclosed herein;

FIG. 4 is an outer shell of the cell culture vessel of FIG. 3 according to some embodiments;

FIG. 5 is an inner container of a cell culture vessel according to another embodiment;

FIG. 6 is a bottom of a shell of the cell culture vessel of FIG. 5 according to some embodiments;

FIG. 7 is a top of the shell of the cell culture vessel of FIG. 5;

FIGS. 8-10 show an expandable dip tube attached to a cell culture vessel according to some embodiments, with FIG. 8 showing an outer tube attached to the vessel, FIG. 9 showing the dip tube in an extended position, and FIG. 10 showing the dip tube in a retracted position;

FIGS. 11-14 illustrate extraction of fluid from a cell culture vessel according to some embodiments; and

FIG. 15 is a schematic representation of a computer system according to one embodiment.

### DETAILED DESCRIPTION

Cancer immunotherapy, including cell-based therapies, are used to provide immune attack of tumor cells while sparing normal cells. As part of cell therapy, immune cells may be obtained for a subject to be treated, the cells may be genetically engineered for administration, and then the cells may be administered (e.g., introduced or re-introduced into the subject) for treatment. Alternatively, cells may be selected or expanded without (or with) genetic modification.

In some embodiments, the immune cells may be autologous (obtained from the same patient) or allogenic (obtained from a donor of the same species as the recipient). In some embodiments, prior to introduction into the subject, the immune cells may be genetically engineered for expression of certain factors. For example, in one illustrative embodiment, prior to introduction in the subject, the immune cells (e.g., T lymphocytes) may be activated or expanded ex vivo. As another example, in embodiments in which the immune cells are allogenic, the allogenic T lymphocytes may be engineered to reduce graft-versus-host effects or host-versus graft effects. For example, expression of the endogenous T cell receptor may be inhibited or eliminated prior to introduction in the subject. As will be appreciated, the cells may be activated and/or expanded by any suitable methods known in the art.

In some embodiments, the genetic engineering step (e.g., preparation of the cell therapy) may include cell separation, cell growth, and analysis. For example, in some embodiments, a cell culture may be maintained in a cell culture vessel in an incubator to allow the cells to grow. In some embodiments, the cell culture vessel may be removed from the incubator, and the cells may be separated from the fluid in the cell culture, such as via centrifugation. For example, in one embodiment, after centrifugation, a cell culture may include a plasma layer, a white blood cell layer, a ficoll layer, and a red blood cell layer.

In some embodiments, media and/or one or more buffers may be added to the cell culture vessel. For example, media and/or buffer(s) may be added to the cell culture to promote cell growth and allow for expansion of the volume of the cells as the cells proliferate. In some embodiments, fluid (e.g., the ficoll layer) may be extracted from the cell culture before adding media and/or buffer(s). In some embodiments, fluid may be extracted from the cell culture after analyzing the cell culture but before adding media and/or buffer(s). As will be appreciated, fluid need not be removed prior to adding media and/or buffer(s) to the cell culture. As will be further appreciated, the cell culture need not be analyzed prior to adding media and/or buffer(s). For example, the cell culture vessel may be simply removed from the incubator and media and/or buffer(s) may be added to the cell culture. In some embodiments, after the desired amount of media and/or buffers is added to the cell culture, the cell culture vessel may be returned to the incubator until it is time for another cell separation and/or addition of media and/or buffer(s). In some embodiments, the cells may be separated prior to introduction or reintroduction, as the case may be, of the cells into the subject.

Typically, the cell therapy is prepared using individual devices, also referred to herein as workstations, and manual operation. For example, a technician may remove the cell culture vessel from the incubator, add fresh media and buffer to the cell culture, and return the cell culture vessel to the incubator for further cell growth. In some examples, the cells may be separated from the liquid in the culture and a sample may be extracted before adding new buffer(s) and media to the culture. The cells also may be analyzed before separation and/or adding the buffer(s) and media. For example, a technician may take a sample from the cell culture to analyze the cells prior to adding buffer(s) and/or media. In some embodiments, the buffer may include a viral vector carrying a transgene.

The inventors have recognized that advantages may be achieved by having an automatable manufacturing system that is configured for high-volume production of cell therapies, e.g., to produce cell therapies for multiple subjects at the same time or sequentially. In some embodiments, this may include producing a cell therapy for a first subject at the same time a cell therapy for a second subject is produced. As will be appreciated, the process step for each of the cell therapies need not happen at the same time. For example, a first cell culture may be analyzed while a second cell culture is being separated and/or that buffer(s) and/or media is being added to a third cell culture. In another example, the second cell culture may be analyzed after the first cell culture is analyzed. The inventors have also recognized that existing high-volume manufacturing systems do not provide a satisfactory solution in all aspects (e.g., batch variability).

The inventors have also recognized that advantages may be realized by having a high-volume manufacturing system with a single workstation that performs the same type of unit operation on various cell cultures. For example, the manufacturing system may have a first workstation that performs sampling and analysis of a cell culture and a second workstation that separates the cells from the liquid. In some embodiments, this may allow a first cell culture to be processed at the same time, or sequentially after, a second cell culture. Advantages also may be achieved if such a system were automated. For example, a robotic device (e.g., a mechanical arm) may move the cell culture vessels between the incubator and various workstations, as will be described.

The inventors have also recognized that advantages may be realized if the same cell culture vessel may be used throughout the entire genetic engineering step, whether part of an automated manufacturing system or a manually operated system. For example, advantages may be realized, if a single cell culture vessel may be transported back and forth between the incubator and the different workstations, with fluid being removed from and added to the cell culture vessel, until the cell therapy is ready for introduction, or reintroduction, into the patient. In some embodiments, the cell therapy may be stored in the cell culture vessel after manufacture is complete. Applicant has recognized that existing cell culture vessels do not provide a satisfactory solution in all aspects.

Without wishing to be bound by theory, cells may need a certain cell density in a cell culture vessel in order for the cells to properly grow. As such, if a cell culture vessel large enough to accommodate a final volume of the cell therapy, e.g., 1 to 2 liters of fluid, were used at the start of the genetic engineering process when there may only be 50 ml of fluid in the cell culture vessel, the inventors have recognized that the cells in the cell culture may not grow. Thus, the inventors have recognized that advantages may be realized by providing a cell culture vessel with a volume that is adjustable during manufacture. As will be described, in some embodiments, such a cell culture vessel may include a container with one or more subsections that may be segregated during manufacture.

### I. Cell Culture Vessel

The inventors have recognized that many advantages including solving the above mentioned problems, may be realized if the cell culture vessel included an assembly with an inner cell culture container (also referred to herein as “inner container”), and an outer shell. Accordingly, one embodiment relates to a cell culture vessel comprising an inner container having a pocket defining a volume within which a cell culture is maintained during manufacture of a cell therapy, and an outer shell arranged to receive and support the container, wherein the outer shell includes a shell top and a shell bottom that cooperate with one another to form a chamber within which the inner container is disposed, optionally, encapsulated. In some embodiments, the inner container may simply contain the cell culture during manufacture and may only include little functionality such as receiving the inner container and thereby supporting it during the transit between different cell manipulation steps. In some embodiments, the inventors have recognized that such an inner container may be easily fabricated, e.g., a flexible cell culture bag from thin plastic materials, which may keep costs down for consumers, may increase the ease of getting and using the cell culture vessel, and may simplify the manufacturing process as well as reduce plastic waste. For example, the inner containers may be disposable, and may be easily loaded into the outer shell during preparation of a first cell therapy, and switched out when a second cell therapy is to be prepared. In turn, such disposable inner container would fit into a reusable outer shell, which provides the support for the bag to transfer the cell culture bag between incubator and work stations. In one embodiment, the outer shell fits into the rotor of a centrifuge and thereby can the cell suspensions be directly centrifuged allowing for automation, as the outer shell can be easily grabbed by a robot to be transferred into and from the centrifuge.

The inner cell culture container may include any container suitable for containing the cell culture (e.g., flexible bags). For example, the inner container may be formed of rigid materials, flexible materials, deformable materials, stretchable materials, or combinations thereof. In some embodiments, the inner container may include an inner bag arranged to contain the fluid. In other embodiments, the inner container may include a rigid frame and one or more film components attached to the frame. In some embodiments, the inner container may be formed, at least in part, of a gas permeable film to allow oxygen diffusion for cell growth and have one or more conduits for fluid transfer.

In some embodiments, the volume of the pocket in any of the cell culture vessels disclosed herein is adjustable, preferably by one or more clamps comprised by the outer shell. In an exemplary embodiment, the volume of the pocket, arranged to maintain the cell culture during manufacture of the cell therapy, is adjustable, optionally, wherein the volume of the pocket is adjustable via a clamp, which optionally is a sliding clamp.

In some embodiments, the pocket includes first, second, and third subsections; and the first subsection is arranged to be segregated from the second and third subsections such that only the first subsection maintains the cell culture during a first portion of the manufacture, optionally, wherein the third subsection is arranged to be segregated from the first and second subsection such that the first and second subsections maintain the cell culture during a second portion of the manufacture. In other embodiments, the pocket includes first, second, and third subsections; and the outer shell is arranged to engage with the inner container to form each of the first, second, and third subsections, optionally, wherein the outer shell includes first and second clamps, the first and second clamps arranged to engage with the container to form the first, second, and third subsections. Accordingly, cell cultures may be initially grown in a small, segrated volume of the inner container, whereas other compartments of the inner container can be added by removing prepositioned clamps without the need for invasion of the inner container.

The inventors have further recognized that advantages may be realized if the cell culture vessel (e.g., culture bag) is well positioned and protected while maintaining sterility of its contents (e.g., cell culture, vector, media etc.). In some embodiments, the outer shell may be arranged to support and/or protect the inner container. Accordingly, the inner container is designed to align in the outer shell for such support and/or protection. For example, the protective shell may help maintain the shape of the container, or at last a portion of the container, during manufacture. In some instances, the interior surface of the outer shell is a rigid cavity, such that an external surface of the inner container is configured to align and mate with the interior surface of the outer shell, when the inner container is placed within the rigid cavity. In some embodiments, the shell may protect the container during travel to and/or processing at one of the workstations. For example, in embodiments in which the container includes a bag, the shell may protect the bag from puncturing or rupturing when stress is applied on the bag, such as during centrifugation. In some embodiments, the outer shell further comprises at least one opening to align the inner container.

The outer shell also may be arranged to interact with the inner container and/or the cell culture during manufacture. For example, the shell may cooperate with the container to create subsections in the container for holding the cell culture as the cells grow. The shell also may encourage mixing of the cell culture in the container. For example, the shell may be arranged to at least partially compress part of the container to encourage mixing of the cell culture. In some embodiments, the shell also may be arranged to sense and/or measure certain characteristics of the cell culture in the container. For example, in some embodiments, the cell culture vessel may be arranged for non-invasive sensing. In such embodiments, the shell may include one or more single-use sensors, such as an optical, pH, or capacitance sensor.

In some embodiments, the outer shell further comprises at least one channel, optionally a tubing port. Although the cell culture container has been described as being arranged to only contain the cell culture in some embodiments, in other embodiments, the container also may include one or more of the above-described single-use sensors (e.g., optical, pH, or capacitance sensors) to sense certain characteristics of the cell culture. The container also may be able to encourage mixing of the cell culture according to some embodiments.

The inventors have also recognized that advantages may be realized if the cell culture vessel (e.g., the assembly of the inner container and the outer shell) had one or more connectors arranged to connect the cell culture vessel to a workstation (or to another container or cell processing device). Advantages also be realized if the cell culture vessel included one or more conduits, e.g., tubing, such as a dip tube, that may allow for fluid transfer into and out of the cell culture vessel, or at least a part of the vessel. The inventors have further recognized that advantages may be realized if the shell was arranged to align the tubing of the cell culture container in the shell such that the container and shell may be properly connected to a workstation (e.g., via an operator or via a robotic device) or to another device, fluid source, or sampling container.

In some embodiments, the cell culture vessel includes a cell culture vessel assembly with an inner cell culture container and an outer shell. In some embodiments, the containers are single-use containers. In some embodiments, the inner container is disposable. For example, the inner container may include single-use bags that are insertable into the outer shell. In such embodiments, the outer shell may be reusable. In some embodiments, at least a portion of the container includes a gas permeable film that allows for oxygen diffusion into the vessel to promote cell growth. In such embodiments, at least a portion of the outer shell may allow for airflow into the shell.

In some embodiments, the outer shell is arranged to support and/or protect to the inner container. In some embodiments, the shell may protect the inner container from puncturing and/or tearing during transport and/or connection of the cell culture vessel (e.g., the assembly) to a workstation. In some embodiments, the shell also may provide support for the inner container during processing. For example, the shell may provide support when stress is exerted on the container, such as during centrifugation, which may prevent rupturing of the container, or at least a portion of the container.

In some embodiments, the cell culture container includes a pocket within which the cell culture is contained during manufacture. In some embodiments, a volume of the pocket of the container may be adjusted during the manufacturing process. For example, in some embodiments, the pocket may be arranged to have a smaller volume during the start of manufacturing. In such an example, the volume of the pocket may be increased as manufacturing progresses and the volume of the cell culture increases with cell growth.

In some embodiments, the container may be at least partially pressed, pinched or otherwise clamped to reduce a volume of the pocket during at least part of the manufacturing process. One or more unused portions of the container also may be rolled up to reduce the volume of the pocket. For example, the container may be pressed, pinched, clamped and/or rolled up to segregate a smaller volume of the pocket for use during part of the manufacturing process. In such embodiments, the container may be partially unrolled and/or the container may be clamped, pressed, or pinched at a different location to increase the volume of the pocket for use during subsequent parts of the manufacturing process, such as when the cell culture volume increase. As will be appreciated, the container may be fully unrolled, unclamped, unpressed, and/or unpinched to return the container to its original volume at the end of manufacture. In some embodiments, the volume of the bag is 50 ml to 5 L. In some embodiments, the volume of the bag is between 0.5 to 5 L. In some embodiments, the volume of the bag is 20 L.

In some embodiments, the cell culture vessel may have one or more conduits for extracting fluid from the cell culture vessel and/or transferring fluid into the cell culture vessel. In some embodiments, the inner container includes one or more conduits arranged to transfer fluid into and out of the pocket, optionally wherein the one or more conduits are attached to a first end of the inner container. For example, in some embodiments, fluid (e.g., a ficoll layer) may be extracted before media and/or buffer(s) is/are added to the cell culture vessel. In some embodiments, as will be described, the conduits may be attached to a first end of the container and may extend into the pocket of the container. In some embodiments, the conduits extend to different depths in the pocket of the container. For example, each conduit may extend into the pocket a different distance relative to the first end of the container and/or pocket. In such examples, by extending different depths in the pocket, each conduit may be arranged to extract different portions of the fluid in the cell culture vessel. For example, a first conduit may be arranged to extract the ficoll layer while a second conduit may be arranged to extract the red blood cell layer. In specific embodiments, the first conduit extends to a first depth in the pocket and the second conduit extends to a second depth in the pocket, the second depth being different from the first depth. In other embodiments, the first conduit extends in a first subsection of the inner container and the second conduit extends in a second subsection of the inner container. As will be appreciated, in other embodiments, the cell culture vessel may include a single conduit, which may be arranged to extend to the different depths in the pocket (e.g., different distances relative to the first end of the container and/or pocket). In some embodiments, the one or more conduits includes one or more dip tubes, wherein the one or more dip tubes includes a first dip tube. In some embodiments, a portion of the dip tube is collapsible. In some embodiments, the first dip tube is extendable into and retractable out of the pocket of the container, optionally, wherein the first dip tube is arranged to move at least one of up and down, side to side, and in a circle to mix the cell culture in the pocket. In some embodiments, the one or more conduits are received in one or more channels in the outer shell.

In some embodiments, the outer shell may have one or more elements to encourage mixing of the cell culture in the pocket of the inner container. In some embodiments, the shell and/or the cell culture vessel may include one or more sensors to analyze the cell culture. In exemplary embodiment, the shell top includes an opening through which liquid in the container may be sensed, optionally wherein the shell includes a sensor arranged to sense the cell culture.

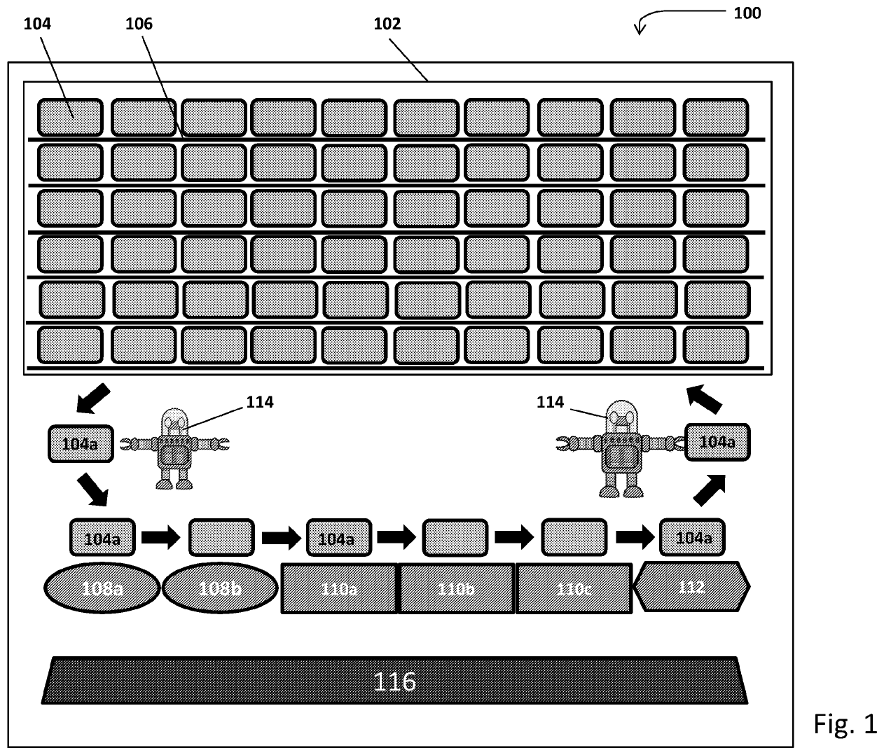

FIG. 1 shows a schematic representation of a manufacturing system according to embodiments disclosed herein. In some embodiments, the system 100 may include a bioreactor or incubator 102 configured to house one or more cell culture vessels 104. In some embodiments, the cell culture vessels 104 may be arranged on one or more shelves 106 or racks. As will be appreciated, although 10 culture vessels are shown on each of the 6 shelves, the incubator may include more or less vessels per shelf. As will be further appreciated, although the incubator is shown as housing 60 culture vessels, the incubator may be arranged to house any suitable number of culture vessels. For example, the incubator may house 5, 10, 20, 50, 100, or more culture vessels.

As will be appreciated, the cell culture vessel may be any suitable shape or size and have any suitable format. For example, in some embodiments, the cell culture vessel may include a dish, glass, cup, or bag, or combinations thereof. In some embodiments, as will be described, the cell culture vessel may include an assembly with an inner container and an outer shell. For example, in some embodiments, the assembly may include an inner bag and an outer shell. The cell culture vessel may be arranged to remain closed, except for when the cell culture vessel is being processed at one of the workstations or there is another fluid transfer into and/or out of the cell culture vessel.

In some embodiments, each of the cell culture vessels is indexed for tracking. For example, the culture vessel 104 may include a tag, chip, label, or other identifier arranged to track the location and progress of the culture vessel in the system. In some embodiments, the identifier may be a visual identifier, such as number or barcode printed on an outside of the culture vessel. In other embodiments, the identifier may include an RFID tag with electronically-stored information. As will be appreciated, any suitable identifier may be used to track the culture vessel in the system. In some embodiments, the system is arranged to read and decode the tag (e.g., scan the barcode and/or read the RFID tag) when the cell culture vessel reaches one of the workstations. The system also may be arranged to read the tag when the cell culture vessel is leaving the workstation (e.g., at exit). In this regard, each of the workstations may include a reader for reading the identifier (e.g., tag) on the cell culture vessel. In embodiments having an automated system, the robotic devices (e.g., robots) also may include a reader for reading the identifier.

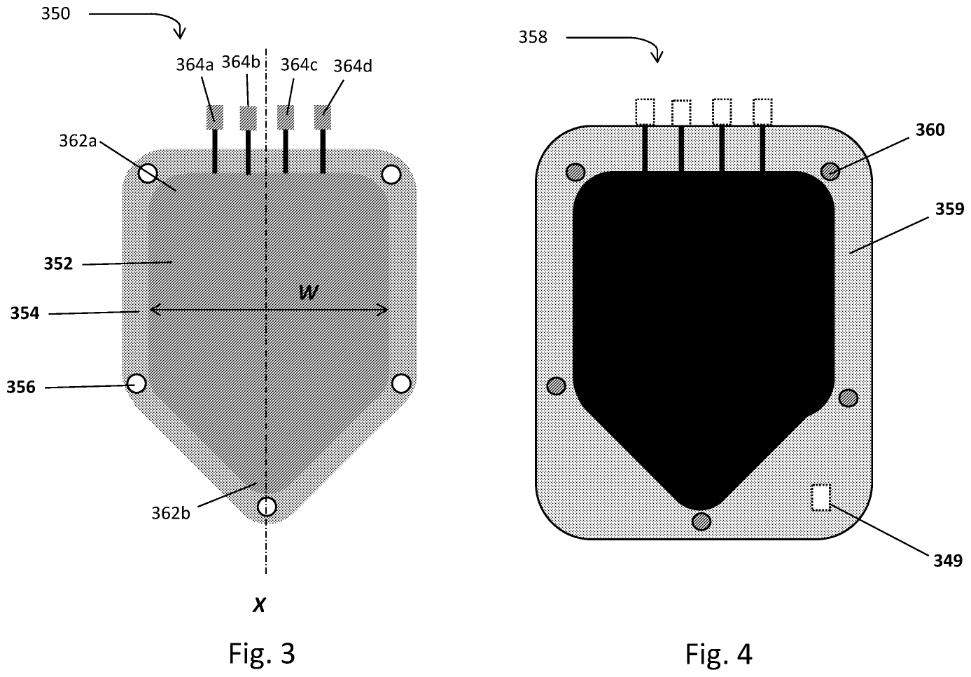

In some embodiments, the identifier is printed on, embedded in, or otherwise integrally formed with the culture vessel. In other embodiments, the identifier may be attached to the culture vessel before placement in the incubator. For example, the tag may be attached to the outer shell within which the inner container is placed before the culture vessel is inserted into the incubator. See, for example, FIG. 4, where a tag, or chip 349 is shown on the shell. As will be appreciated, the tag or chip also may be located on the inner container in some embodiments. A tag or chip also may be located on both the container and the outer shell.

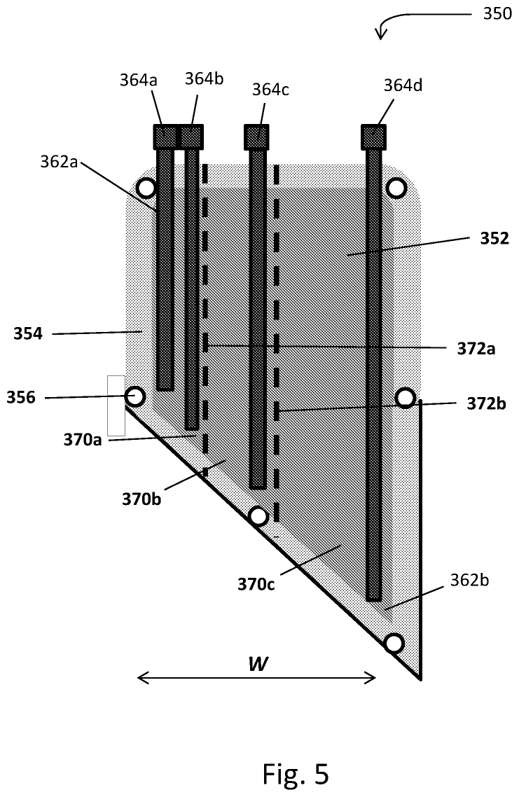

According to one aspect of the present disclosure, a cell culture vessel may include an assembly with an inner cell culture container, such as a cell culture bag, and an outer shell. FIGS. 3 and 5 illustrates examples of a cell culture container 350 and FIGS. 4 and 6-7 illustrate examples of an outer shell according to embodiments of the present disclosure. In some embodiments, the container may not include any sensors and/or may not be capable of processing any information. In some embodiments, the container is arranged to be used only once, for preparation of a single cell therapy. In such embodiments, the outer shell may be reusable.

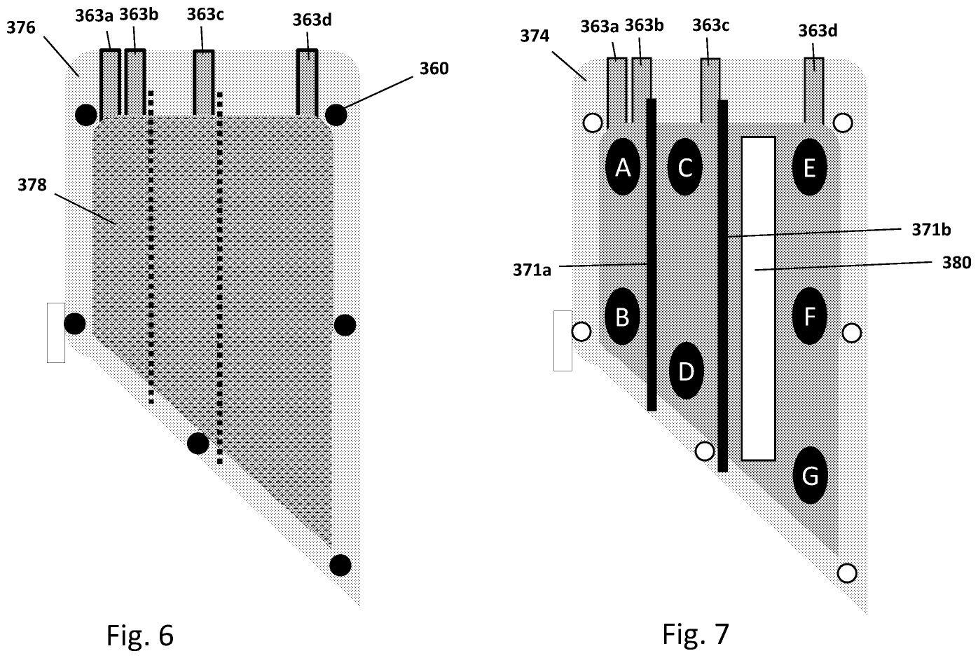

As shown in FIGS. 6 and 7, the shell may include a shell top 374 and shell bottom 376 that cooperate to form a chamber into which the cell culture container is housed during manufacture of the cell therapy. As will be appreciated the top and bottom of the shell may be attached to one another during manufacture of the cell therapy, and then detached from one another when the container is ready to be removed, such as when the cell therapy is to be administered. In some embodiments the top and bottom of the shell may be hingedly attached to one another. In some embodiments, the top and bottom of the shell include top and bottom halves of the shell. As will be appreciated, the top and bottom of the shell need not be the same shape and size, although the top and bottom of the shell may be the same shape and size in some embodiments. In some embodiments, the shell top is removably attachable to the shell bottom.

In some embodiments, as shown in FIGS. 3 and 5, the container may include an inner pocket 352 within which the cell culture is maintained during manufacture. In some embodiments, the container is sealed such that the cell culture cannot escape from the pocket unless extracted by an extraction device (e.g., via a conduit during a fluid transfer step). In some embodiments, as shown in FIGS. 3 and 5, a flange 354 may extend around the pocket, forming an outer edge of the container. In some embodiments, the flange may be formed via heat sealing.

In some embodiments, the flange of the container may include one or more openings 356 arranged to align the container within the outer shell 358. For example, the outer shell may have a frame 359 with one or more alignment pins 360 that may cooperate with the one or more openings to align the container in the shell. In some embodiments, the pins may be slid through the openings to align the container in the shell.

As will be appreciated, although the container and shell in FIGS. 3 and 4 are shown with five openings and five corresponding alignment pins, respectively, the container may have one or more openings and the shell may have one or more corresponding alignment pins in other embodiments. For example, the container and shell in FIGS. 5 and 6-7 have six openings and six alignment pins, respectively. In such embodiments, the openings and corresponding pins may be positioned at any suitable locations around the flange and around the shell, respectively. As will be further appreciated, although the container is shown as having openings and the shell is shown as having alignment pins, in other embodiments, the container may have one or more alignment pins and the shell may include one or more openings into which the pins may be inserted. The openings and corresponding alignment pins may have any suitable cross-sectional shape and size. As shown in FIGS. 3 and 4, in some embodiments, the cross-sectional shape of the alignment pins and the opening may be circular, although the pins and openings may be square, triangular, oval, or other cross-sectional shape in other embodiments.

As shown in FIGS. 3 and 5, in some embodiments, the pocket may include a width W that decreases from a first end 362a of the pocket towards the second end 362b of the pocket. In such embodiments, the first end of the pocket may include an end of the pocket to which one or more fluid conduits may be attached. In some embodiments, the width of the pocket decreases from the first end to the second end. In such embodiments, a width of the pocket at the second end may be smaller than a width of the pocket at any other location between the first and second ends (and including the first end).

In some embodiments, the width of the pocket may decrease gradually from the first end to the second end. In some embodiments, as shown in FIGS. 3 and 5, the width may taper gradually from a portion of the pocket between the first and second ends to the second end of the pocket. In some embodiments, as shown in FIG. 3, the width of the pocket also decrease may from the right and left sides of the pocket to a central region, such as to a point equidistant between the first and second sides of the pocket. In some embodiments, as shown in FIG. 5, the width of the pocket may decrease from a left side of the pocket to the right side of the pocket. The width of the pocket also may decrease from the right side of the pocket to the left side in other embodiments, as will be appreciated. In some embodiments, the pocket of the inner container and the outer shell includes a width that decreases in a direction from a first end of the pocket towards a second end of the pocket, optionally wherein the width of the pocket is smallest at or near the second end pocket and/or wherein a second end of the pocket is triangular in shape. In some instances, the inner container having the pocket is an angled cell culture bag. In other stances, the cell culture bag has sloped bottom.

In some embodiments, as shown in FIG. 3, the pocket may be symmetric about a longitudinal axis X of the pocket. In other embodiments, the pocket may be asymmetric about the longitudinal axis X (see, e.g., FIG. 5).

Although the pocket is shown as having a width that is not uniform between the first and second ends of the pocket, in other embodiments, the width of the pocket uniform from the first end of the pocket to the second end of the pocket. As will be appreciated, the shape and size of the pocket need not be the same as the shape and size of the container. For example, in some embodiments, the pocket may have a width that decreases between the first and second ends of the pocket, while the container may have a width that is uniform between the first and second ends of the container.

As shown in FIGS. 3 and 5, the second end of the pocket may have a substantially triangular shape in some embodiments. In such embodiments, the second end also may have a slanted bottom. In some embodiments, the triangular shape and slanted bottom of the pocket may allow for sedimentation of the cells in the cell culture. In some embodiments, as shown in FIG. 5, such a triangular shape may result in a corner of the container being the lowest point during centrifugation. Without wishing to be bound by theory, in some embodiments, this may allow for tighter packing of the cells.

In some embodiment, the pocket may be divided into subsections having smaller volumes. For example, as shown in FIG. 5, in some embodiments, the pocket may be divided into three subsections 370a, 370b, 370c to allow the cell culture to grow in smaller volumes to maintain a desired cell density for optimal growth. As shown in this view, each of the first, second and third subsections may include a triangular shape and slanted bottom at the second end. As will be appreciated in view of the above, this shape may allow for sedimentation of the cells in the cell culture in each of the subsections during manufacture.

Although three subsections are shown in FIG. 5, it will be appreciated that the cell culture container may have more or fewer subsections. For example, the container may have two subsections in some embodiments, or may have four or more subsections in other embodiments. As will be appreciated, the shape and size of the subsections may be the same in some embodiments, although they may differ in other embodiments.

In some embodiments, the shell may engage with the container to divide the pocket into the different subsections. For example, in some embodiments, the case may have clamps 371a, 371b (see FIG. 7) arranged to clamp down on the container (see the dashed lines labeled 372a, 372b in FIG. 5) to create the subsections 370a, 370b, 370c in the pocket. In an illustrative embodiment, at the start of manufacturing, the first clamp may be pressed down on the container (at line 372a), the clamp being pressed against the shell bottom, to establish the first subsection 370a for holding the cell culture. As the cells grow and the fluid in the pocket expands beyond a volume that the first subsection can accommodate, the second clamp may press down on the container (at line 372b) to create the second and third subsections, and then the first clamp may be released to allow fluid to fill the first and second subsections. In a similar fashion, as the cells continue to grow, the second clamp may be released so that the full volume of the pocket (e.g., the first, second, and third subsections) may be accessible to hold the cell culture.

Although the first subsection is described as being the first to receive the cell culture during manufacture, it will be appreciated that the second and/or third subsections may be the first to receive the cell culture during manufacturing in other embodiments. In such embodiments, the respective clamp or clamps may engage with the container to segregate off the second or third subsection. As the cell culture grows, one or more of the remaining subsections may be accessed to allow the cell culture to flow into two, and later all of the subsections.

In some embodiments, the clamps may be arranged to open and close automatically. In some embodiments, operation of the clamps is directed by the controller 116. For example, the clamps may move according to a set manufacturing schedule. The clamps also may open in response to sensed data. An operator also may manually operate the clamps in some embodiments. In some embodiments, the clamps move relative to the outer shell. For example, as will be appreciated, when the shell is secured around the container, the clamps may move in a direction towards a center of the shell to clamp the container at one of the noted areas. In such an example, the clamps may move in a direction away from the center of the shell to release the container at one of the noted areas.

Although clamps are shown and described for separating the pocket into various subsection, it will be appreciated that other suitable arrangements may be used. For example, in another embodiment, the shell may include a roller which may roll back and forth on the container to segregate off the desired subsection(s) in the pocket. In another example, unused portions of the container may be rolled up to segregate off desired subsection(s) in the pocket. The container also may be pinched off at different locations (e.g., via a pincher on the shell) to segregate off smaller volumes in the pocket for the cell culture to grow.

In some embodiments, as shown in FIGS. 3 and 5, the cell culture container may include one or more conduits 364a-d arranged to transfer fluid into and out of the pocket of the container. In some embodiments, as shown in FIG. 3, the conduits may not extend into the pocket. As shown in FIG. 5, in other embodiments, each of the conduits may extend into the pocket. In some embodiments, as shown in FIG. 5 the conduits may extend to different depths into the pocket. In such embodiments, the conduits may be located different distances from the first end of the pocket. For example, the first conduit 364a may extend partially between the first and second ends 362a, 362b of the pocket, while the fourth conduit 364d may extend to a position at or near the second end 362b of the pocket. As will be described, the locations of the conduits in the pocket may allow for extraction of different types of fluids from the cell culture vessel.

In some embodiments, as shown in FIG. 5, one or more conduits may extend into each of the subsections. For example, the first two conduits 364a, 364b may extend into the first subsection 370a, the third conduit 364c may extend into the second subsection 370b, and the fourth conduit 364d may extend into the third subsection 370c. Without wishing to be bound by theory, this may allow for extraction of fluid from or transfer of fluid into one or more of the subsections, no matter the stage of manufacturing of the cell culture and the subsection(s) being used during that stage. As will be appreciated, each of the subsections may include the same number of conduits, although the number of conduits in each subsection may vary from subsection to subsection.

In some embodiments, the conduits may include dip tube. In some embodiments, the dip tube may include a needless cannula. The dip tube also may include an aseptic fitting or another interface arranged for automation.

In some embodiments, when the container is aligned in the shell, the fluid conduits of the container may be aligned with and held in channels 363a-d formed in the shell (see FIGS. 6 and 7). In some embodiments, the top and bottom of the shell may cooperate with one another to form each of the channels 363a-d within which the conduits 364a-d of the container are received. In some embodiments, the channels may facilitate proper attachment of the container to a workstation (e.g., via a connector).

Although the container is shown as having four fluid conduits in this embodiment, it will be appreciated that the container may have one or more fluid conduits in other embodiments for fluid transfer. In some embodiments, the conduits may be integrally formed with the container. In other embodiments, the conduits may be removably attachable to the container. For example, the conduits may be inserted into the container via outlets formed in a side wall prior to inserting the cell culture into the container, and/or inserting the cell culture container into the outer shell.

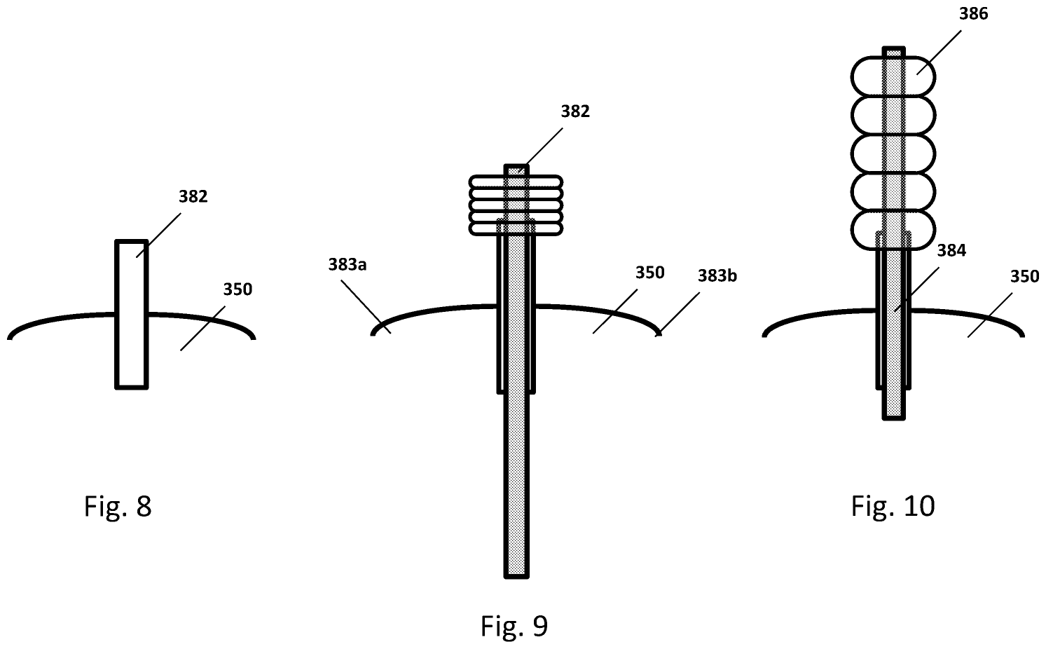

Although the container is shown and described as having multiple conduits extending to different depths in the pocket, in other embodiments, as illustrated in FIGS. 8-10, the container may include a single dip tube that is extendable into container (e.g., to different depths in the pocket) and retractable out of the container. FIG. 8 illustrates the container 350 with an installed outer tube 382. FIG. 9 illustrates the dip tube 384 positioned within the outer tube, the dip tube being in an extended position inside the container. FIG. 10 illustrates the dip tube 384 being retracted out of the container. As shown in FIG. 10, when the tube is retracted, the tube may be covered by a cover 386. As shown in FIGS. 9 and 10, the tube cover may be collapsible (e.g., like an accordion) when the dip tube is again extended into the container. As will be appreciated, the container may have more than one extendable dip tube in other embodiments.

In some embodiments, the dip tube may be moveable in a linear direction, e.g., back and forth between first and second sides 383a, 383b of the pocket. As will be appreciated, in some embodiments, the dip tube may be moved between the different subsections to extract fluid at different stages of manufacture of the cell culture.

In some embodiments, the shell may be arranged to immobilize the dip tube during manufacture of the cell therapy, such as at one of the first and second sides of the pocket or at a location in between the first and second sides of the pocket. For example, the shell may halt linear travel of the dip tube in the container, such as during a certain stage of manufacture. In some embodiments, the shell may immobilize the tubing to determine the location of the dip tube relative to the shell. Such localizing of the dip tube may allow use of one or more robotic devices, such as for automated systems.

In some embodiments, the retractable dip tube may be arranged for stirring and/or mixing of the cell culture. For example, in some embodiments, the dip tube may be actuated and repeatedly moved up and down in the pocket, or in a subsection of the pocket, of the container. In some embodiments, the distal end of the dip tube may be formed with a molded, flared end. In some embodiments, such as when the dip tube is inserted into a smaller subsection of the pocket, the flared end may fill more of the volume and may be able to create flow and minimize abrasion. As will be appreciated, the distal end of the tube may have other suitable arrangements in other embodiments. In some embodiments, such as when the dip tube is inserted into a larger volume subsection, or in the entire pocket, the dip tube may be swung back and forth, like a pendulum, to provide mixing. Such a swinging motion may be accomplished via a flexible or V-shaped top port. As will be appreciated, the dip tube may be moved in other directions or patterns (e.g., in a circular movement) to mix the cell culture within the pocket.

In some embodiments, mixing by the dip tube may be performed automatically, such as with an automated system. In such embodiments, the dip tubes may be actuated via the controller. Mixing by the tip tube also may be performed manually.

As shown in FIGS. 6 and 7, in addition to having the clamps, the shell may have other features arranged to engage with and/or manipulate the container. For example, the top 374 of the shell may include one or more mixing members A-G to facilitate mixing of the cell culture in the container. In some embodiments, the first and second mixing members A, B may encourage mixing of fluids in the first subsection of the pocket, such as at the beginning of manufacturing. Mixing members C, D may encourage mixing of fluids in the second subsection and mixing members E, F, G may encourage mixing of fluids in the third subsection. As will be appreciated, in some embodiments, mixing members A-D may encourage mixing of fluids in the first and second chamber of the pocket, such as at a later point during manufacture. In other embodiments, any or all of the mixing members A-G may be used to encourage mixing of fluids housed in the entire pocket.

In some embodiments, the mixing members may include plungers that may be actuated and moved up and down to compress the container and cause the fluid in the container to move around and mix. In some embodiments, the plungers may be moveable relative to the top 374 of the shell. In some embodiments, the plungers also may be moveable relative to one another. For example, all the plungers need not move in the same direction at the same time. In some embodiments, half of the plungers may move in an upward direction while the other half of the mixing members move in a downward direction. The plungers also may be moveable up and down in other compression sequences. For example, the plungers may be arranged to move up and down in pairs. The plungers also may be activated to move in a circular pattern.

In some embodiments, the plungers may be arranged to compress the container the entire way (e.g., move to the bottom of the shell). In other embodiments, the plungers may only partially compress the container. In some embodiments, the shell may be controlled by the controller and may operate a compression scheme to move the mixing members and mix the fluid in the vessel.

As will be appreciated, although the plungers are described as moving up and down (e.g., translating back and forth), the plungers also may be arranged to move in other directions. For example, the plungers may be arranged to translate and rotate. The plungers also may be arranged to translate and pivot back and forth.

As will be appreciated, in some embodiments, mixing by the dip tube may be performed in addition to mixing via the mixing members on the shell. In some embodiments, mixing by the dip tube may be performed at a different time than mixing via the mixing members. For example, mixing by the dip tube may be alternated with mixing via the mixing members. In some embodiments, mixing by the dip tube may happen at the same time as mixing with the mixing members.

In some embodiments, as shown in FIG. 6, at least a portion 378 of the bottom 376 of the shell may include mesh or perforations, or be supported on mesh or ridges to allow air flow into and out of the shell. Although only the bottom of the shell is arranged to allow airflow into and out of the shell in FIGS. 6 and 7, in other embodiments, the top of the shell also may be arranged to allow airflow into and out of the shell.

In the embodiment shown in FIG. 7, the top of the shell may be formed of a solid material, except for the where the mixing members A-G may be located. As will be appreciated, in such locations, the top of the shell may include openings for receiving the mixing members, the mixing members moving up and down in the openings as the mixing members compress the container.

In some embodiment, as shown in FIG. 7, the top of the shell may include a widow or opening 380 for optical sensing. In some embodiments, the shell may include a sensor arranged to sense, and in some embodiments analyze, the cell culture in the container through the window or opening. In other embodiments, the sensor may be located at one of the workstations.

In some embodiments, the top and bottom of the shell may be formed of a rigid material. In some embodiments, the top and bottom of the shell may be formed of a material that is cleanable. In some embodiments, the shell may be formed of stainless steel or polycarbonate material.

In some embodiments, at least a portion of the container, such as a bottom portion of the container that is positioned adjacent to the bottom of the shell, may be formed of a material that is gas permeable or may include a film that is gas permeable. For example, the container may be formed of or include a film that is made of polyolefin. As will be appreciated, the entire container need not be made of the same material. For example, the bottom of the container may be gas permeable while the top of the container is formed of a different, stronger material, or even of a rigid material. For example, in some embodiments, the top of the container may be reinforced to be able to withstand the compressions by the plunger to mix the cell culture in the container and the clamping of the container by the clamps to create the different subsections of the container. In some embodiments, the container may be formed of a rigid frame with one or more flexible components, such as film components. In some embodiments, the films may be positioned adjacent the top and bottom of the shell to allow for compression via the mixing members and to allow for oxygen transfer into the container.

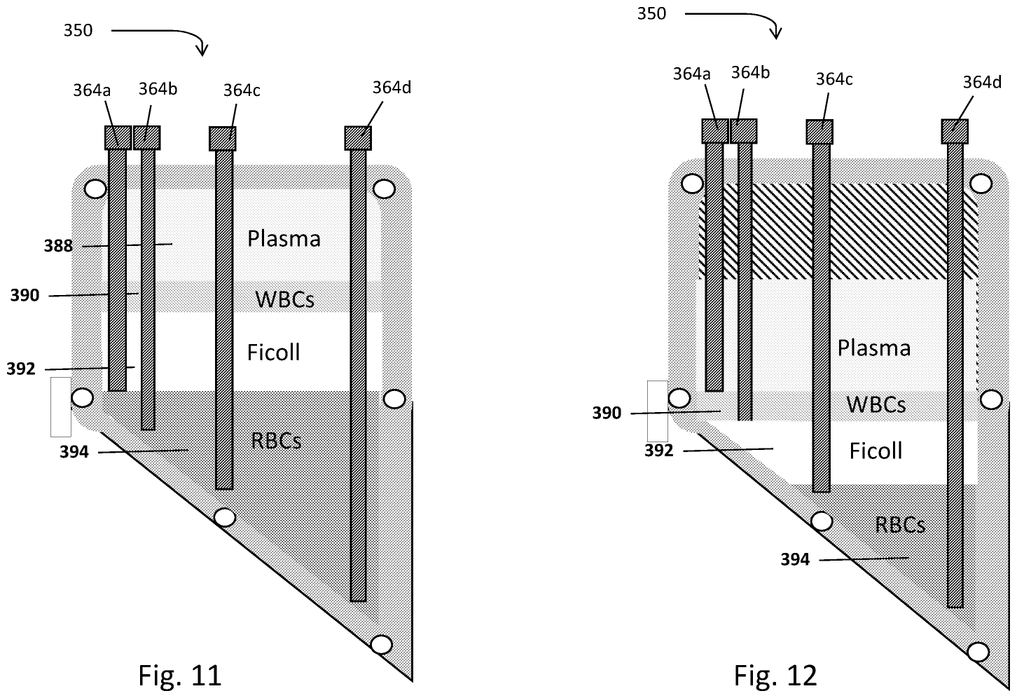

FIGS. 11-14 illustrate use of the cell culture container, such as for extraction of the one or more fluids from the cell culture. As shown in FIG. 11, after cell separation, such as centrifugation, the cell culture may be separated into four discrete layers in the pocket of the container. For example, the cell culture may include a plasma layer 388, a white blood cell layer 390, a ficoll layer 392, and a red blood cell and granulocyte layer 394. As will be appreciated, the layers may be separated based on the weight of the layer. For example, the plasma layer may the top layer while the red blood cells and granulocyte layer is the bottom layer.

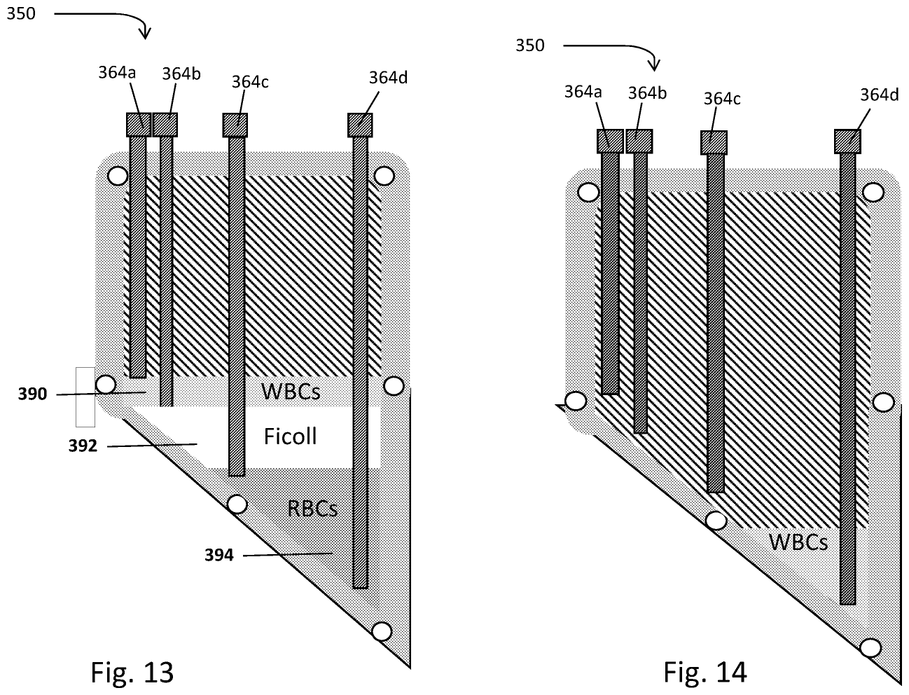

Next, as illustrated in FIG. 12, the red blood cells may be removed via the fourth dip tube 364d until the bottom of the plasma layer aligns with the bottom of the first dip tube 364a. As will be appreciated, removal of the red blood cells may be controlled manually, or may be automated via pre-set volumes or optical level sensors. In one such example, optical sensors may be enabled by density-specific dies for the waste layer (e.g., Ficoll layer) and/or for specific cell stains.

The plasma layer may then be removed via the first dip tube 364a. FIG. 13 illustrates the container after the plasma layer and part of the red blood cell layer has been removed. Finally, the remaining red blood cells and the Ficoll layer may be removed via the fourth dip tube 364d until the bottom of the white blood cell layer approaches the bottom of the fourth dip tube (see FIG. 14).

In some embodiments, the white blood cells may be washed and separated after the above-noted steps. In some embodiments, the white blood cells may be washed and separated until a desired purity of the white blood cells is achieved.

As will be appreciated, although the layers have been shown and described as being extracted until a layer reaches the position of a specific dip tube in the culture container, in other embodiments, the layers may be extracted via a volumetric directive from the controller (or by an operator). For example, the controller may instruct the workstation to extract a prescribed volume of white blood cells from second dip tube 364b after the first red blood cells are removed from the container and the plasma layer reaches the bottom of the first dip tube 364a (see e.g., FIG. 12). In other embodiments, removal of the desired T-cells may be done using a weighed measurement.

In some embodiments, the white blood cells may be stored in the container after extraction of the other fluids from the container. In such embodiments, after the white blood cells are washed and the purity of the white blood cells is established, the container may be returned to the incubator for storage. The white blood cells may then be removed from the container for introduction, or reintroduction, into a patient.

Although a single cell culture vessel is shown and described for use in a manufacturing system to prepare a cell therapy, in other embodiments, more than one cell culture vessels may be used to prepare the cell therapy. For example, in some embodiments, cell culture containers with pockets having different volumetric sizes may be inserted into the shell and used for the various stages of manufacture. In some embodiments, a container with a pocket having a smaller volume (e.g., similar in size to that of the first subsection) may be inserted into the outer shell during the first stages of manufacture. When the volume of the cell culture exceeds the volume of the first pocket, the cell culture may be transferred to a second container with a pocket having a larger volume, the second container being insertable into the shell. As with the first container, the cell culture can be removed from the second container and inserted into a third container having a pocket with an even larger volume, as cell growth continues. The third container may then be inserted into the shell. In some embodiments, the volume of the pocket of the container is chosen so as to maintain a desired cell density in the cell culture vessel.

### II. Manufacturing System

According to one aspect of the present disclosure, a manufacturing system may include one or more workstations used to prepare a cell therapy. In some embodiments, the manufacturing system includes more than one of the same type of workstation. For example, a manufacturing system may include two analysis workstations. As will be appreciated, the manufacturing system need not have the same number of workstations for each unit operation. For example, the manufacturing system may include two analysis workstations, three separation workstations, and a single liquid addition workstation. In some embodiments, the manufacturing system may be customized depending upon the cell therapies being produced and the process steps used to produce those cell therapies. For example, in some embodiments, the separation step make take more time than the step of adding media and buffer(s). Thus, the manufacturing system may include more separation workstations such that the manufacturing system can efficiently process more samples at the same time, or in sequence. In other embodiments, the analysis step may be the rate limiting step (e.g., the step that takes the longest time), and the system may include more analysis workstations than separation workstations. In some embodiments, this customization of the number and type of workstations may allow the manufacturing system to efficiently utilize all workstations to produce a large volume of cell therapies. As with the above, in some embodiments, each of the workstations may be removable and/or replaceable if repairs are needed or upgrades are available.

According to another aspect of the present disclosure, the manufacturing system may be automated. For example, the inventors have recognized that advantages may be achieved by having a high-volume manufacturing system that requires little to no technician input. In one example, the system may include one or more robotic devices, such as robots or robotic arms, arranged to move the cell culture vessel between the incubator and one or more of the workstations.

In some embodiments, the manufacturing system may include an incubator for storing one or more cell cultures. In some embodiments, each of the cell cultures may belong to a different subject. For example, the incubator may include 100 cell cultures belonging to 100 different subjects. The incubators also may include more than one cell culture per subject. For example, the incubator may include 100 cell cultures belonging to 50 different subjects (e.g., two cell cultures per subjects). In such embodiments, the cell cultures may be labeled and indexed. For example, each culture may have an identification number corresponding to the subject and cell culture sample. In some embodiments, the manufacturing system includes a process controller, such as a computer, which may store the identification number and track the progress of the cell cultures in the manufacturing system. For example, the controller may track the location of the cell culture in the incubator and/or in one of the workstations. The controller also may control operation of the various workstations and incubator.

In some embodiments, the manufacturing system includes connectors arranged to connect the workstation to one or more cell culture vessels. As will be appreciated, the connectors may be directly attached to the workstation in some embodiments, although, in other embodiments, the connectors may be removably attached to the workstations. In some embodiments, each workstation may include one connector arranged to connect each cell culture vessel to the workstation, although the workstation also may include more than one connector to connect each cell culture vessel to the workstation. In some embodiments, each workstation may have the same type of connector, while in other embodiments, the connector may vary from workstation to workstation. For example, a connector arranged to connect the cell culture vessel to a separation workstation may differ from a connector arranged to connect the cell culture vessel to an analyzing or a liquid transfer workstation. As will be further appreciated, the workstation also may be arranged to connect more than one cell culture vessel to the workstation at the same time.

In some embodiments, the connector may be attached to the cell culture vessel. For example, the connector may be attached to the outer shell of the cell culture vessel assembly. In some embodiments, the connector may travel with the cell culture vessel between workstations to connect the cell culture vessel to the workstations. As with the above, the connector may be permanently attached to the cell culture vessel or may be removably attached to the cell culture vessel.

In some embodiments, the connector may include a tube or conduit that may be connected to a tube or conduit attached at the workstation. For example, the connector may be attached to tubing used to transfer fluid into and/or out of the workstation. In some embodiments, the connector also may be attached to a tube or conduit attached to the cell culture vessel. For example, the connector may allow for transfer of fluid into and/or out of the cell culture vessel. As will be appreciated, the connector may allow for fluid transfer between the cell culture vessel and another container, such as a sampling container or a fluid source.

As shown in FIG. 1, the manufacturing system may include one or more workstations for processing the cell culture. In some embodiments, the system may include one or more workstations for sampling and analytics. For example, at the sampling and analyzing workstation 108a, 108b, a sample may be withdrawn from the cell culture vessel and one or more tests may be performed on the sample, as will be appreciated by those of skill in the art. For example, the workstation may determine the level of cell growth or whether or not additional time in the incubator is needed before administering the cell therapy to the subject.

In some embodiments, the sampling and analyzing workstation may include one or more pipettes arranged to withdraw the sample from the culture vessel. As will be appreciated, other suitable arrangements may be used to withdraw the sample from the culture vessel. For example, in some embodiments, the culture vessel may be connected to the workstation via a connector, such as a conduit or tube. In such embodiments, the workstation may be arranged to withdraw the sample via the conduit or tube. For example, the workstation may apply a vacuum to draw the sample into the conduit or tube and into the workstation for testing. The workstation also may draw a sample into the conduit and then into a separate testing container (e.g., a bag, tip, pouch), which may be transferred to another different workstation (or to an off-site location) for testing.

In some embodiments, each of the sampling and analyzing workstations may perform the same test. In such embodiments, a first culture vessel may be processed by only one of the sampling and analyzing workstations 108a, 108b before being moved to a second workstation (e.g., the separation work station). As will be appreciated, in such embodiments, a second cell culture vessel may be processed at the second sampling and analyzing workstations before being moved to another workstation (e.g., one of the separation work stations). In other embodiments, each of the sampling and analyzing workstations may perform different tests. In such embodiments, the first culture vessel may be moved to both the first and second sampling and analyzing workstations before moving to another workstation (e.g., one of the separation workstations). In such embodiments, the second culture vessel may be processed at each of the first and second sampling and analyzing workstations after the first culture vessel is processed.

As shown in FIG. 1, after visiting one of the sampling and analyzing workstations, the cell culture vessel may be moved to another type of workstation, such as to one of the separation workstations 110a, 110b, 110c. As with the above, in some embodiments, the cell culture vessel may be moved to only one of the separation workstations, although cell culture vessel also may be moved to more than one of the separation workstations before moving along to another workstation. In some embodiments, at the separation workstation, the cells are separated from the liquid medium in the culture vessels. In some embodiments, the separation workstation may include a centrifuge. The separation workstation also may include an acoustic separator.

In some embodiments, the separation workstation is arranged to separate the cell culture into discrete layers, such as via centrifugation. For example, the cell culture vessel may be separated into a white blood cell layer, a red blood cell layer, a ficoll layer, and a plasma layer (see, e.g., FIG. 11). In some embodiments, the separation workstation is also arranged to remove the liquid medium from the culture vessel. For example, similar to the sampling and analyzing workstation, the separation workstation may include a pipette for removing liquid medium (e.g., the ficoll or plasma) from the culture vessel. In such embodiments, the culture vessel also may be connectable to the separation workstation via a connector such that liquid medium may be drawn into the conduit and into the workstation.

After removing the liquid medium at one of the separation workstations, the cell culture vessel is moveable to a liquid addition workstation 112. In some embodiments, one or more buffers, viruses, or media may be added to the cell culture vessel. The liquid may be added to the cell culture vessel via any suitable method, such as via a pipette or via a conduit connected the vessel to the workstation. As will be appreciated, other suitable liquids may be added to the cell culture vessel at the liquid addition workstation 112. After adding the one or more liquids, the cell culture vessel may be returned to the incubator 102 to allow the cells to continue growing.

As shown in FIG. 1, in some embodiments, the manufacturing system may be automated and may include one or more robotic device, such as robots 114, that transfer the cell culture vessels to and from the workstations. For example, the robot may bring the cell culture vessel from the incubator 102 to one of the sampling and analyzing workstations 108a, 108b, then to one of the separation workstations 110a-c, and then finally to the liquid addition workstation 112, before returning the cell culture vessel to the incubator. As will be appreciated, the robot also may include a reader arranged to read the identifier on the cell culture vessel before and after the vessel is delivered to one of the workstations and the incubator.

Although the system is shown as having robots for moving the vessel between the different workstations and incubator in FIG. 1, in other embodiment, the cell culture vessel may move between the workstations via other suitable methods. For example, in some embodiments, the cell culture vessel may move between workstations via a conveyor belt. The manufacturing system also may be manually operated, such that a user (e.g., a technician) may move the cell culture vessels between the workstations and incubator.

In some embodiments, the system may include a controller 116 arranged to control each of the components of the manufacturing system 100. For example, the controller may control the temperature and carbon dioxide (CO₂) level of the incubator. The controller also may direct the robots to move culture vessel between the incubator and the different workstations according to a desired schedule, if the system is automated. For example, the robots may be programmed to move the cell culture vessel through the manufacturing system according to a desired schedule. In some embodiments, the culture vessel may be moved through the various workstations every hour, every couple of hours, every day, and/or after several days. In some embodiments, the schedule may be determined based on the cell therapy being prepared. In some embodiments, the schedule may be modified based on dynamic feedback received from the workstations. For example, the scheduled may be varied (e.g., increased or decreased) based on the calculated cell count. In some embodiments, the controller 116 also may control each of the workstations to process the cell culture.

In some embodiments, the controller may be arranged to collect and store data from each of the workstations during the manufacturing process. In some embodiments, the controller is arranged to process the collected data. The controller also may be arranged to adjust the operating schedule and/or one or more operating parameters of one of the workstations based on the feedback of another workstation. For example, in some embodiments, the amount of medium added to the cell culture vessel may be based on the cell count. In such embodiments, based on the measured cell count, the volume of medium to be added to the cell culture vessel may be adjusted.

Generally, the controller comprises a memory circuit storing instructions and a processor circuit configured to execute the instructions and/or a memory circuit storing data of the manufacturing operations and sampling. In some instances, a controller may be programmed to perform the steps automatically. In some embodiments, the controller comprises a memory circuit and a processor circuit, the memory circuit storing instructions which, when executed by the processor circuit, cause preceding embodiments to be performed automatically. The system can comprise a computing device (CPU) which can be in communication with a data storage device. In an embodiment, the data storage device can store system data and at least one operating parameter. The data storage can be in the same location as the CPU or at an offsite location wherein the CPU is in telecommunication with the data storage system. The system can further comprise a plurality of sensors, the sensors can comprise measuring devices that are configured to provide data to the CPU regarding the operation of each component within the system. The sensors displayed in the system can include but not limited to position sensors, pressure sensors, optical sensors, temperature sensors, force sensors, vibration sensors, piezo sensors, fluid property sensors, time sensors and/or humidity sensors. The system can comprise these sensors to provide data to the CPU to initiate and maintain operation of the system. The data received from the sensors located at the various components of the systems provided data to automate a continuous feedback loop that permits the CPU to maintain and adjust the operation of all components of the system. In a workstation, for example, at a defined timepoint, a controller directs a robot to move a specific container (for e.g., culture bag) comprising a tissue culture (cell container). The robot further puts the inner container within the outer shell. The robot aligns the inner container within interior surface of the rigid cavity of the outer shell. The alignment is carried with respect to openings comprised within the outer shell.

The controller conducts the claimed method of manufacturing one or more cell therapies using a system having an incubator and one or more workstations. The controller further moves the cell culture vessel to a first workstation where at least one of separating the cell culture, analyzing the cell culture, and transferring liquid into and out of the cell culture vessel and removing the cell culture vessel from the first workstation. The controller via a robot may further maneuver the outer shell that includes a shell top and a shell bottom, the shell top and shell bottom cooperating with one another to form a chamber within which the container is disposed, optionally, encapsulated. The robot may further align conduits of the inner container to a fixed position. The controller compares the cell count value a with predefined value/range. If the measured value is too low, the controller controls the robot to return the cell container to the incubator and reschedules the cell container for the next manipulation step according to a preset algorithm. If the measured value is at the defined value or higher, the controller controls the robot to place the cell container and/or with the outer shell into a centrifuge and controls the centrifuge to centrifuge the cells at a given speed and time. After the centrifugation the controller controls the robot to move the cell container and/or with the outer shell back to the device and puts the tube connected to a waste container. The controller may repeat the method for manufacturing one or more cell therapies (e.g., addition of specific feeds, cytokines, isolation & analysis of cell types) according to programs predefined or by values transmitter from sensors to the controller programmed to undertake specific cell manipulations for specific values. Once all of the defined cell manipulation steps have been completed, the controller directs the robot to remove the cell container from the outer shell and return it to the incubator at a predefined position or at a random empty position, which is then stored by the controller.

In some embodiments, the controller may include a computer or computer system. In some embodiments, the controller may include a tablet or other mobile electronic device (e.g., a mobile telephone). In some embodiments, the controller is connected to each of the workstations and to the incubator. As will be appreciated, the controller may be connected to these devices via any suitable connection, such as via the internet, Ethernet, wireless, Bluetooth, or other suitable connection.

In some embodiments, the controller may control workstations based on one or more desired operating parameters. For example, the controller may direct the separation workstation to run a centrifuge for a desired period of time and/or may direct the liquid addition workstation to add a prescribed volume of a buffer to the cell culture vessel. In some embodiments, the operating parameters are determined based upon the cell therapy being prepared. As will be appreciated, the operating parameters may vary from cell therapy to cell therapy.

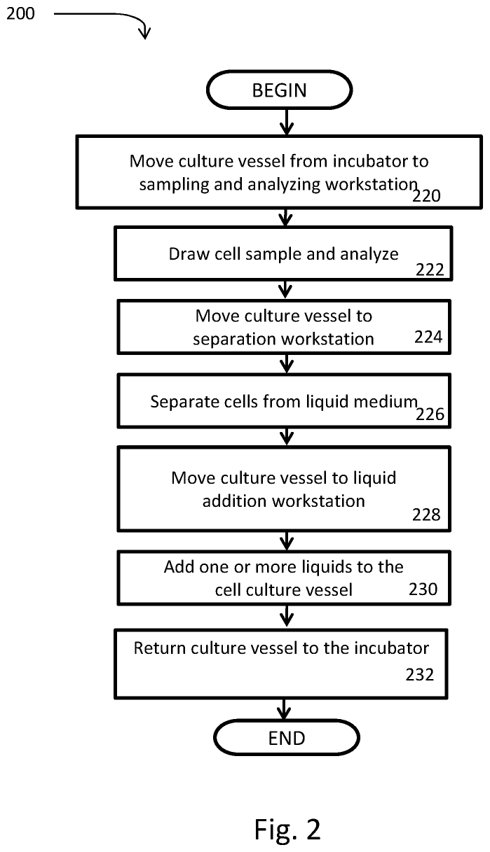

An illustrative example of a manufacturing process 200 of a manufacturing system of a cell therapy is shown in FIG. 2. As shown in this figure, the process may include moving the culture vessel from the incubator to the sampling and analyzing workstation 220, drawing a sample of the cell culture, and analyzing the sample 222. Next, the process may include moving the culture vessel to the separation workstation 224, where the cells may be separated from the liquid medium 226, such as via a centrifuge. The cell culture vessel may then be moved to the liquid addition workstation 228, where one or more liquids may be added to the cell culture vessel 230. Finally, the culture vessel may be returned to the incubator 232.

As will be appreciated, in embodiments in which the system is automated, the controller may direct one or more robotic devices to perform the steps shown in FIG. 2, such as moving the cell culture vessel to the different workstations. The controller also may collect and evaluate data during processing. In some embodiments, one or more of the process steps may be skipped or altered depending upon dynamic feedback. As will be further appreciated, each of the steps also may be performed manually.

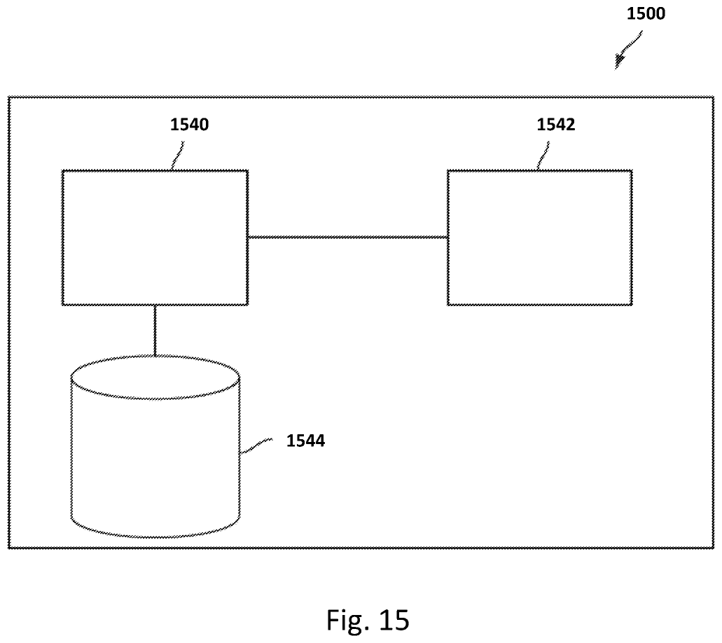

FIG. 15 shows an illustrative implementation of a computer system 1500 that may be used in connection with some embodiments of the present disclosure. One or more computer systems, such as computer system 1500, may be used to implement any of the functionality described above. The computer system 1500 may include one or more processors 1540 (e.g., processing circuits) and one or more computer-readable storage media (i.e., tangible, non-transitory computer-readable media), e.g., volatile storage 1542 (e.g., memory) and one or more non-volatile storage media 1544, which may be formed of any suitable non-volatile data storage media. The processor(s) 1540 may control writing data to and reading data from the volatile storage 1542 and/or the non-volatile storage device 1544 in any suitable manner, as aspects of the present disclosure are not limited in this respect. To perform any of the functionality described herein, processor(s) 1542 may execute one or more instructions stored in one or more computer-readable storage media (e.g., volatile storage 1544), which may serve as tangible, non-transitory computer-readable media storing instructions for execution by the processor 1540.

### III. Method for Manufacturing

An embodiment is a method of manufacturing one or more cell therapies using a system having an incubator and one or more workstations wherein the method comprises moving a cell culture vessel to a first workstation, wherein the cell culture vessel comprises an inner container having a pocket defining a volume within which a cell culture is maintained during manufacture of a cell therapy, and an outer shell at least one of separating the cell culture, analyzing the cell culture, and transferring liquid into and out of the cell culture vessel; and removing the cell culture vessel from the first workstation. In some embodiments, the inner container is designed to align in the outer shell. In some embodiments, wherein the outer shell includes a shell top and a shell bottom, the shell top and shell bottom cooperating with one another to form a chamber within which the container is disposed. As described above, the cell culture vessel according to the invention provides numerous advantages for being applied in the manufacturing of cell therapies

In some embodiments, the method further comprises robotic maneuvering to align conduits of the inner container to a fixed position, optionally the conduits are openings, optionally the conduits of the inner container are received by outer channels in the outer shell. It is especially important for automated methods that channels for manipulation of the cells are in fixed positions, so that robots can easily be programmed to conduct the respective manipulation step. Accordingly, the conduits of the inner container are aligned to the channels in the outer shell.

In some embodiments, the method comprises (i) removing the cell culture vessel from an incubator arranged to house one or more cell culture vessels; (ii) moving the cell culture vessel to the first workstation, and/or (iii) adding a cell culture into the pocket of the container, optionally, wherein the step of adding includes adding the cell culture into a first subsection of the pocket of the container, the container having first, second and third subsections. In some embodiments, the method comprises segregating the first subsection from second and third subsections, optionally wherein segregating the first subsection includes clamping a first portion of the container with a first clamp of the shell to segregate the first subsection from the second and third subsections before the step of adding the cell culture into a first subsection of (iii). In some embodiments, the method comprises (i) actuating the first clamp via a controller before the step of clamping, and/or (ii) mixing the cell culture, optionally, wherein mixing the cell culture includes moving one or more conduits in the pocket of the container. In some embodiments, the method comprises (i) at least partially compressing part of the container with one or more mixing members on the outer shell; and/or (ii) actuating the one or more mixing members via a controller.

### IV. General Remarks

The above-described embodiments of the present disclosure can be implemented in any of numerous ways. For example, the embodiments may be implemented using hardware, software or a combination thereof. When implemented in software, the software code (e.g., instructions) can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above. In some embodiments, the control of unit operations may be performed via an integrated 3rd party software or control on a particular device, while a global system (e.g., SCADA) may be provided for supervisory control, data acquisition, and/or scheduling.

In this respect, it should be appreciated that one implementation of embodiments of the present disclosure comprises at least one computer-readable storage medium (i.e., at least one tangible, non-transitory computer-readable medium, e.g., a computer memory, a floppy disk, a compact disk, a magnetic tape, or other tangible, non-transitory computer-readable medium) encoded with a computer program (i.e., a plurality of instructions), which, when executed on one or more processors, performs above-discussed functions of embodiments of the present disclosure. The computer-readable storage medium can be transportable such that the program stored thereon can be loaded onto any computer resource to implement aspects of the present disclosure. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-discussed aspects of the present disclosure.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Also, the disclosure may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “including” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, the term “connected” is defined as attached, whether directly or indirectly through intervening components, and is not necessarily limited to physical connections. The connection can be such that the objects are permanently connected or releasably connected.

It should also be understood that, unless clearly indicated to the contrary, in any methods claimed herein that include more than one step or act, the order of the steps or acts of the method is not necessarily limited to the order in which the steps or acts of the method are recited.

The invention is also described by the following items:
1. A cell culture vessel comprising:
   an inner container having a pocket within which a cell culture is maintained during manufacture of a cell therapy; and
   an outer shell arranged to receive the container, wherein the shell includes a shell top and a shell bottom that cooperate with one another to form a chamber within which the container is disposed.
2. The cell culture vessel of item 1, wherein the container is disposable.
3. The cell culture vessel of item 2, wherein the shell is reusable.
4. The cell culture vessel of any one of items 1-3, wherein a volume of the pocket arranged to maintain the cell culture during manufacture of the cell therapy is adjustable.
5. The cell culture vessel of item 4, wherein the volume of the pocket is adjustable, which optionally is via a sliding clamp.
6. The cell culture vessel of item 4 or item 5, wherein:
   the pocket includes first, second, and third subsections; and the first subsection is arranged to be segregated from the second and third subsections such that only the first subsection maintains the cell culture during a first portion of the manufacture.
7. The cell culture vessel of item 6, wherein the third subsection is arranged to be segregated from the first and second subsection such that the first and second subsections maintain the cell culture during a second portion of the manufacture.
8. The cell culture vessel of item 4 or item 5, wherein:
   the pocket includes first, second, and third subsections; and
   the shell is arranged to engage with the container to form each of the first, second, and third subsections.
9. The apparatus of item 8, wherein the shell includes first and second clamps, the first and second clamps arranged to engage with the container to form the first, second, and third subsections.
10. The cell culture vessel of any one of items 1-9, wherein the pocket of the container includes a width that decreases in a direction from a first end of pocket towards a second end of the pocket.
11. The cell culture vessel of item 10, wherein the width of the pocket is smallest at or near the second end pocket.
12. The cell culture vessel of item 10 or item 11, wherein a second end of the pocket is triangular in shape.
13. The cell culture vessel of any one of items 10-12, wherein the pocket is symmetric about a longitudinal axis.
14. The cell culture vessel of any one of items 10-12, wherein the pocket is asymmetric about a longitudinal axis.
15. The cell culture vessel of any one of items 1-14, wherein the container includes one or more conduits arranged to transfer fluid into and out of the pocket.
16. The cell culture vessel of item 15, wherein the one or more conduits are attached to a first end of the container.
17. The cell culture vessel of item 15 or item 16, wherein the one or more conduits include first and second conduits, wherein the first conduit extends to a first depth in the pocket and the second conduit extends to a second depth in the pocket, the second depth being different from the first depth.
18. The cell culture vessel of any one of items 15-17, wherein the one or more conduits includes first and second conduits, wherein the first conduit extends in a first subsection of the container and the second conduit extends in a second subsection of the container.
19. The cell culture vessel of any one of items 15-18, wherein the one or more conduits includes one or more dip tubes.
20. The cell culture vessel of item 19, wherein the one or more dip tubes includes a first dip tube, wherein the first dip tube is extendable into and retractable out of the pocket of the container.
21. The cell culture vessel of item 20, wherein the first dip tube is arranged to move at least one of up and down, side to side, and in a circle to mix the cell culture in the pocket.
22. The cell culture vessel of any one of items 19-21, wherein the one or more dip tubes includes a first dip tube, wherein the first dip tube is moveable between a first side of the pocket and a second side of a pocket.
23. The cell culture vessel of item 22, wherein the shell cooperates with the container to stop travel of the first dip tube at one or more of the first side of the pocket, the second side of the pocket, and a position in between the first and second sides of the pocket.
24. The cell culture vessel of any one of items 15-23, wherein the one or more conduits are received in one or more channels in the shell.
25. The cell culture vessel of any one of items 1-24, wherein the shell top is removably attachable to the shell bottom.
26. The cell culture vessel of any one of items 1-25, wherein the shell bottom includes at least one of a mesh or perforations arranged to allow airflow into and out of the shell.
27. The cell culture vessel of any one of items 1-26, wherein the shell includes one or more mixing members arranged to encourage mixing of the cell culture vessel in the pocket of the container.
28. The cell culture vessel of item 27, wherein the one or more mixing members include one or more plungers.
29. The cell culture vessel of item 28, wherein the one or more plungers are moveable relative to the shell top.
30. The cell culture vessel of item 29, wherein the one or more plungers are moveable relative to one another.
31. The cell culture vessel of any one of items 1-30, wherein the shell top includes an opening through which liquid in the container may be sensed.
32. The cell culture vessel of item 31, wherein the shell includes a sensor arranged to sense the cell culture.
33. The cell culture vessel of any one of items 1-32, wherein:
   the container includes a flange extending around the pocket, the flange having one or more openings; and
   the shell bottom includes one or more corresponding alignment pins arranged to be inserted into the one or more openings to align the container in the shell.
34. The apparatus of any one of items 1-33, wherein at least a portion of the container is gas permeable.
35. The apparatus of any one of items 1-34, wherein at least a portion of the container is formed of a flexible material.
36. The apparatus of item 35, wherein the flexible material includes a film.
37. The apparatus of item 35 or item 36, wherein a least a part of the container is formed of a rigid material.
38. The apparatus of any one of items 1-37, wherein at least one of the container and the shell includes a tag, chip, or tracking label.
39. The apparatus of item 38, wherein at least one of the container and the shell include an RFID tag.
40. The apparatus of any one of items 1-39, wherein the shell is formed of a rigid material.
41. A method of manufacturing one or more cell therapies using a system having an incubator and one or more workstations, the method comprising:
   moving a cell culture vessel to a first workstation, wherein the cell culture vessel includes an inner container having a pocket within which a cell culture is maintained during manufacture of a cell therapy and an outer shell arranged to support the container;
   at least one of separating the cell culture, analyzing the cell culture, and transferring liquid into and out of the cell culture vessel; and
   removing the cell culture vessel from the first workstation.
42. The method of item 41, further comprising, before the step of moving the cell culture vessel to the first workstation, removing the cell culture vessel from an incubator arranged to house one or more cell culture vessels.
43. The method of item 41 or item 42, wherein the outer shell includes a shell top and a shell bottom, the shell top and shell bottom cooperating with one another to form a chamber within which the container is disposed.
44. The method of any one of items 41-43, further comprising adding a cell culture into the pocket of the container.
45. The method of item 44, wherein the step of adding includes adding the cell culture into a first subsection of the pocket of the container, the container having first, second and third subsections.
46. The method of item 45, further comprising, before the step of adding the cell culture into a first subsection, segregating the first subsection from second and third subsections.
47. The method of item 46, wherein segregating the first subsection includes clamping a first portion of the container with a first clamp of the shell to segregate the first subsection from the second and third subsections.
48. The method of any one of items 41-47, further comprising, before the step of clamping, actuating the first clamp via a controller.
49. The method of any one of items 41-48, further comprising mixing the cell culture.
50. The method of item 49, wherein mixing the cell culture includes moving one or more conduits in the pocket of the container.
51. The method of item 50, wherein moving the one or more conduits includes moving the one or more conduits in at least one of an up and down, side to side, or circular motion.
52. The method of item 50 or item 51, further comprising, before the step of moving, actuating the one or more conduits via a controller.
53. The method of any one of items 41-52, further comprising at least partially compressing part of the container with one or more mixing members on the shell.
54. The method of any one of items 41-53, further comprising actuating the one or more mixing members via a controller.

1. A cell culture vessel comprising:
an inner container having a pocket defining a volume within which a cell culture is maintained during manufacture of a cell therapy; and
an outer shell arranged to receive and support the inner container, wherein the outer shell includes a shell top and a shell bottom that cooperate with one another to form a chamber within which the inner container is disposed, optionally, encapsulated.

2. The cell culture vessel of claim 1, wherein the inner container is disposable, and/or wherein, the outer shell is reusable.

3. The cell culture vessel of claim 1, wherein the inner container is designed to align in the outer shell.

4. The cell culture vessel of claim 1, wherein an interior surface of the outer shell defines a rigid cavity, such that an external surface of the inner container is configured to align and mate with the interior surface of the outer shell, when the inner container is placed in the rigid cavity.

5. The cell culture vessel of claim 1, wherein the outer shell further comprises one or more of the following:
(i) at least one clamp to adjust the volume of the inner container;
(ii) at least one opening to align the inner container; and
(iii) channel, optionally, a tubing port.

6. The cell culture vessel of claim 1, wherein the volume of the pocket arranged to maintain the cell culture during manufacture of the cell therapy is adjustable, optionally, wherein the outer shell comprises the at least one clamp and the volume of the pocket is adjustable via the clamp, which optionally is a sliding clamp.

7. The cell culture vessel of claim 6, wherein:
(i) the pocket includes first, second, and third subsections; and
the first subsection is arranged to be segregated from the second and third subsections such that only the first subsection maintains the cell culture during a first portion of the manufacture, optionally, wherein the third subsection is arranged to be segregated from the first and second subsection such that the first and second subsections maintain the cell culture during a second portion of the manufacture; or
(ii) the pocket includes first, second, and third subsections; and
the outer shell is arranged to engage with the inner container to form each of the first, second, and third subsections, optionally, wherein the outer shell includes first and second clamps, the first and second clamps arranged to engage with the container to form the first, second, and third subsections.

8. The cell culture vessel of claim 1, wherein the pocket of the inner container and the outer shell includes a width that decreases in a direction from a first end of the pocket towards a second end of the pocket, optionally wherein the width of the pocket is smallest at or near the second end pocket and/or wherein a second end of the pocket is triangular in shape.

9. The cell culture vessel of claim 8, wherein
(i) the pocket is symmetric about a longitudinal axis; or
(ii) the pocket is asymmetric about a longitudinal axis.

10. The cell culture vessel of claim 1, wherein the inner container includes one or more conduits arranged to transfer fluid into and out of the pocket, optionally, wherein the one or more conduits are attached to a first end of the inner container.

11. The cell culture vessel of claim 10, wherein
(i) the one or more conduits include first and second conduits, wherein the first conduit extends to a first depth in the pocket and the second conduit extends to a second depth in the pocket, the second depth being different from the first depth;
(ii) the one or more conduits includes first and second conduits, wherein the first conduit extends in a first subsection of the inner container and the second conduit extends in a second subsection of the inner container;
(iii) the one or more conduits includes one or more dip tubes, wherein the one or more dip tubes includes a first dip tube, wherein the first dip tube is extendable into and retractable out of the pocket of the container, optionally, wherein the first dip tube is arranged to move at least one of up and down, side to side, and in a circle to mix the cell culture in the pocket; and/or
(iv) the one or more conduits are received in one or more channels in the outer shell.

12. The cell culture vessel of claim 1, wherein
(i) the shell top is removably attachable to the shell bottom;
(ii) the shell bottom includes at least one of a mesh or perforations arranged to allow airflow into and out of the shell;
(iii) the shell includes one or more mixing members arranged to encourage mixing of the cell culture vessel in the pocket of the container;
(iv) the one or more mixing members include one or more plungers, wherein the one or more plungers are moveable relative to the shell top, optionally, wherein the one or more plungers are moveable relative to one another;
(v) the shell top includes an opening through which liquid in the container may be sensed, optionally wherein the shell includes a sensor arranged to sense the cell culture; and/or
(vi) the container includes a flange extending around the pocket, the flange having one or more openings; and the shell bottom includes one or more corresponding alignment pins arranged to be inserted into the one or more openings to align the container in the shell.

13. A method of manufacturing one or more cell therapies using a system having an incubator and one or more workstations, wherein the method comprises:
moving a cell culture vessel to a first workstation, wherein the cell culture vessel comprises an inner container having a pocket defining a volume within which a cell culture is maintained during manufacture of a cell therapy, and an outer shell arranged to receive and support the container, wherein the outer shell includes a shell top and a shell bottom that cooperate with one another to form a chamber within which the inner container is disposed, optionally encapsulated;
at least one of separating the cell culture, analyzing the cell culture, and transferring liquid into and out of the cell culture vessel; and
removing the cell culture vessel from the first workstation.

14. The method of claim 13, wherein the inner container is designed to align in the outer shell.

15. The method of claim 13, wherein an interior surface of the outer shell defines a rigid cavity, such that an external surface of the inner container is configured to align and mate with the interior surface of the outer shell, when the inner container is placed in the rigid cavity.

16. The method of claim 13, wherein the method further comprises robotic maneuvering to align conduits of the inner container to a fixed position,
optionally the conduits are openings,
optionally the conduits of the inner container are received by outer channels in the outer shell.

17. The method of claim 13, further comprising,
(i) removing the cell culture vessel from an incubator arranged to house one or more cell culture vessels
(ii) moving the cell culture vessel to the first workstation, and/or
(iii) adding a cell culture into the pocket of the container,
optionally, wherein the step of adding includes adding the cell culture into a first subsection of the pocket of the container, the container having first, second and third subsections.

18. The method of claim 17, before the step of adding the cell culture into a first subsection of (iii), further comprises segregating the first subsection from second and third subsections, optionally wherein segregating the first subsection includes clamping a first portion of the container with a first clamp of the shell to segregate the first subsection from the second and third subsections.

19. The method of claim 13, further comprising,
(i) actuating the first clamp via a controller before the step of clamping, and/or
(ii) mixing the cell culture, optionally, wherein mixing the cell culture includes moving one or more conduits in the pocket of the container.

20. The method of claim 19, wherein
(i) moving the one or more conduits includes moving the one or more conduits in at least one of an up and down, side to side, or circular motion; and/or
(ii) before the step of moving, the method further comprises actuating the one or more conduits via a controller.

21. The method of claim 13, further comprising
(i) at least partially compressing part of the container with one or more mixing members on the outer shell; and/or
(ii) actuating the one or more mixing members via a controller.
